(19) United States
(12) Patent Application Publication
(10) Pub. No.: US 2005/0261651 A1
(43) Pub. Date: Nov. 24, 2005

(54) Portable package for articles of personal use

(76) Inventors: Viviane Antao Lima, Sao Paulo (BR); Eduardo Cesar Andreo Aledo, Sao Jose dos Campos (BR)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/109,893

(22) Filed: Apr. 19, 2005

(30) Foreign Application Priority Data
Apr. 22, 2004 (BR) PI 0401630-0

(51) Int. Cl. A61F 13/15

(52) U.S. Cl. 604/385.02

(57) ABSTRACT

A portable package for articles of personal use, such as feminine absorbents, comprising a strip (1) containing at least one row of wraps (10) in the form of tubular “pillows” arranged in series, each wrap (10) involving at least one personal use article (2) and being formed by a front wall (11) and a rear wall (12) having longitudinal lateral edges (13) and transversal end edges (14). Each confronting transversal end edge (14) of each two adjacent wraps (10) are united to each other by a joining flap (20), so that the wraps (10) can be folded one over the other.

### FIELD OF THE INVENTION

The present invention relates to a portable package for containing articles of personal use, such as feminine absorbent articles, particularly internal or external absorbent articles, daily use protectors or body fluid absorbing products.

### BACKGROUND OF THE INVENTION

The prior art discloses packages for containing absorbent articles, such as feminine sanitary napkins, such packages typically comprising a bag or the like made from a plastic film or alternatively the package may be constructed from paper or paperboard. The absorbent articles placed in the above described packages are often individually folded and wrapped in a plastic film pouch or paper wrapper to maintain the absorbent article in a sanitary condition prior to use. The pouch is maintained in a closed condition prior to use by sealing the transverse end edges of the pouch using any suitable process, such as heat, ultrasound, adhesive, etc. The longitudinal edges are arranged on top of one another and are usually glued to one another at a selected location. The longitudinal edges however are not sealed to one another along their entire length, to thereby enable the user to more easily open the wrapper.

The conventional packages for absorbent articles described above are intended to be stored at a selected fixed location, e.g. at home or at work. In this manner, when the user needs a napkin the user removes one of the individually wrapped articles from the box and then unwraps the individually wrapped article.

The known packages described above generally contain a number of absorbent articles designed to fulfill the needs of a user for more than one day, usually several days, or in situations requiring more frequent changes. For example, prior art packages may contain eighteen or more such absorbent articles.

Thus, the prior art packages do not enable the user to carry, if necessary, a small number of absorbent articles considered sufficient to fulfill an immediate need or a small supply of articles which will last until the user can again access the supply at home or at work.

In view of the above, presently users have to carry a package containing ten or more units of absorbents, which is an excessive quantity in relation to the user's immediate need. Further, it is inconvenient for a user to carry around a large package, containing a large number of articles, when only a few absorbent articles may be required for the relevant time period (e.g. a day).

In addition to the disadvantages mentioned above, prior art packages which contain a fixed and relatively large quantity of absorbent articles are not sufficiently versatile to comply with the sporadic needs of the user, but rather require the user to carry a supply of articles which is excessive and unnecessary in many situations.

Another disadvantage of prior art packages results from the fact that the individually wrapped articles are often carried loosely inside the user's purse. As described above, the edges of the wrapper containing the article are superimposed and secured over only a portion of their length thereby enabling the user to more easily open the package. However, when the wrapper is carried in a purse or the like, contaminants can find their way into the wrapper thereby contaminating the article prior to use.

### OBJECT OF THE INVENTION

In view of the foregoing, it is an object of the present invention to provide a portable package for personal use articles, such as feminine absorbent articles, particularly feminine sanitary napkins and liners, which allows the user to carry the desired number of units to satisfy the user's immediate needs, said absorbent articles being protected, individually or jointly, in an arrangement which facilitates the portability and accessibility thereof in a purse or any other similar means.

### SUMMARY OF THE INVENTION

In view of the foregoing, the present invention provides a portable package for articles of personal use, such as feminine absorbents including a strip (1) containing at least one row of wraps (10) each wrap (10) containing at least one personal use article (2) each wrap (10) having a front wall (11) and a rear wall (12) having longitudinal lateral edges (13) and transversal end edges (14) united to each other, wherein transversal end edges (14) of two adjacent wraps (10) are coupled to each other by a joining bridge (20) in order to allow the wraps (10) to be folded one over the other and selectively separated from each other.

### BRIEF DESCRIPTION OF THE DRAWINGS

The invention will be described below, with reference to the attached drawings, which set forth exemplary embodiments of the invention and in which:

FIG. 1 is a perspective view of a portable package according to the present invention, the package formed by a strip having two wraps arranged in a row;

FIG. 2 is a perspective view of the package of FIG. 1 with the wraps folded one over the other, in the form of a case;

FIG. 3 is a partial sectional view of the package illustrated in FIG. 1, said section being taken along line III-III of FIG. 1;

FIG. 4 is a cross-sectional view of the package of FIG. 1 taken along line IV-IV of FIG. 1;

FIG. 5 is a perspective view of a second embodiment of the present package, also formed by a strip having three wraps arranged in row;

FIG. 6 is a perspective view of the package of FIG. 5, with the wraps folded one over the other, in the form of a case;

FIG. 7 is a perspective view of a third embodiment of the package according to the present invention, also formed by a strip having, for example, three wraps arranged in row;

FIG. 8 is a sectional view of the package of FIG. 7, with the wraps folded one over the other;

FIG. 9 is a perspective view of a fourth embodiment of the package according to the present invention formed by a strip having an even number of wraps arranged in row;

FIG. 10 is a partially cut perspective view of the package of FIG. 9 when folded in the form of a case; and

FIG. 11 is a view similar to that of FIG. 9, but illustrating the rear side of the present package.

### DETAILED DESCRIPTION OF THE INVENTION

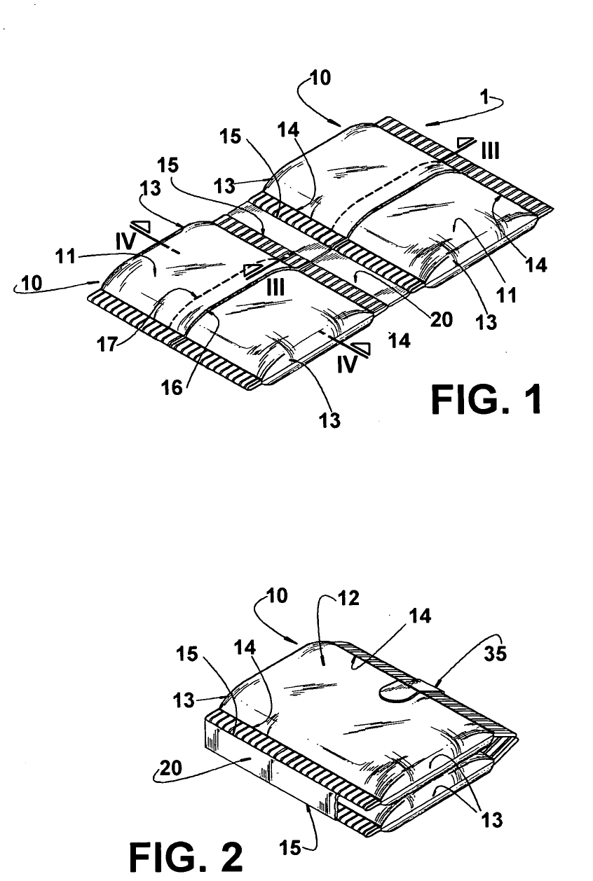

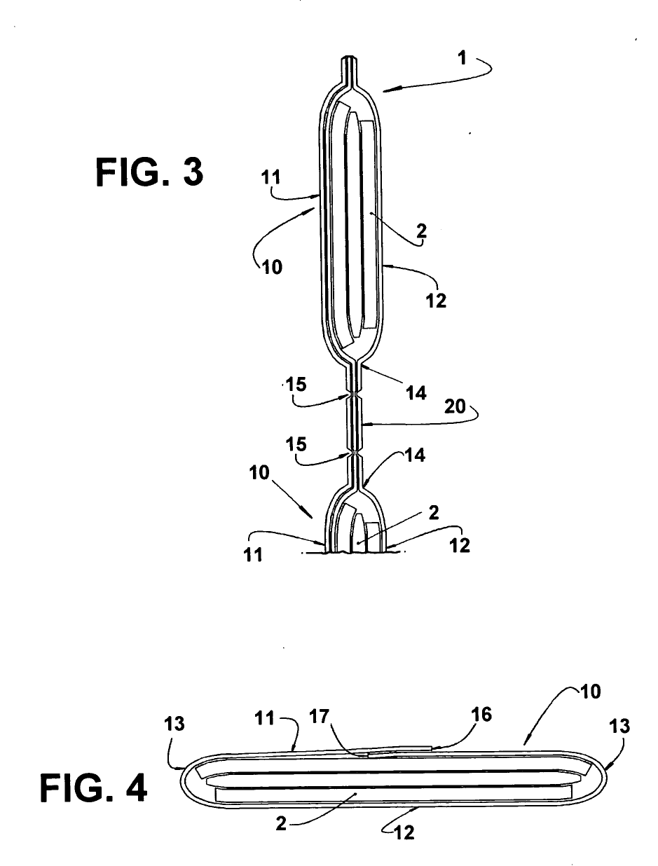

As illustrated in FIGS. 1-4 of the enclosed drawings, the portable package according to the present invention comprises a strip 1 generally having a flat tubular shape and which is made of at least one film of plastic material or paper, or any material suitable for packaging sanitary absorbent articles and the like. Each strip 1 contains at least one row with one or more wraps 10 shaped as tubular “pillows” or containers and arranged in series, each wrap 10 encloses at least one personal use article 2, for example a sanitary napkin or liner. The personal use article is arranged in the wrap 10 in either a flat condition or preferably in a folded condition wherein the article has been folded one or more times as shown in FIGS. 1-4.

Each wrap 10 of a row is formed by a front wall 11 and a rear wall 12, which define longitudinal lateral edges 13 on each side of the wrap 10 and transversal end edges 14 united to each other by heat, ultrasound, etc. The adjacent transversal end edges 14 of two adjacent wraps 10 in the same row are connected to each other by a respective joining flap or bridge 20. The joining bridge 20 is connected to the adjacent transversal end edges 14 of the two adjacent wraps 10 by a respective fold line 15, around which the wraps 10 can be folded in order to occupy positions parallel to each other and orthogonal to the joining bridge 20, defining a case, as illustrated in FIG. 2.

It is possible to construct the package so that at least one of the two fold lines 15 adjacent to a joining bridge 20 includes cutouts and/or weakened lines, that are structured and arranged to be manually ruptured by the user when the user wants to separate a wrap 10 from the row of wraps. This construction allows each wrap 10 to be easily detached from the other wraps 10 of the row, allowing the user to carry in her purse only the number of wraps needed for the intended use.

As illustrated in the drawings, each row of wraps 10 of a strip 1 contains a certain number of wraps 10. The number of wraps 10 contained in a certain row is determined by commercial considerations and also by the number of personal use articles 2 contained inside each wrap.

As illustrated in the figures, particularly in FIGS. 2, 5, 6, 7, 8, 9 and 10, the joining bridges 20 are made of the same material which forms the row of wraps, with the longitudinal edges 13 of the front wall 11 and rear wall 12 being likewise welded to each other.

As shown in FIG. 8, each joining bridge 20 has a length “L”. The selected length “L” of a particular joining bridge is selected based upon the particular folding configuration to be imparted to the wraps 10 (see FIGS. 2, 6, 8 and 10) and in order to approximately correspond to the sum of the thicknesses “E” of the wraps 10 disposed on planes parallel to each other and orthogonal and secant in relation to said joining bridge 20, allowing the wraps to be arranged in a folded condition around the respective fold lines 15 in order to form a compact and reliable package in the form of a case which can be easily accommodated inside a purse or bag.

It should be understood that each strip 1 can be formed by two or more rows of wraps 10 disposed side by side, each row being separated from the immediately adjacent row by a longitudinal fold line (not illustrated), but which is constructed in the same manner as fold lines 15 that divide adjacent wraps 10 arranged in a row.

In order to enable the row of wraps 10 to be easily accommodated inside a purse or bag and carried in the same, the fold lines 15 also define the fold lines around which the wraps 10 can be folded so as to be seated on immediately adjacent wraps 10.

In the construction illustrated in FIGS. 1-4 of the drawings, the wraps 10 have their front wall 11 and rear wall 12 defined by a single continuous film of a packaging material. The packaging material has longitudinal edges 16 and 17 that are arranged in overlapping relationship to one another as best seen in FIG. 1. The overlapping portions of the packaging material are releasably affixed to each other using a suitable adhesive or the like that permits the wrap to be selectively opened and then resealed. This type of construction is particularly useful where each wrap 10 contains two or more personal use articles 2. In this case, after removing a personal use article 2 from a wrap 10 by opening the longitudinal edges 16, 17 these same longitudinal edges 16, 17 can be resealed to each other so as to maintain the remaining personal use article(s) 2 protected inside the respective wrap, until it is removed by the user.

The resealable structure of the wrap 10 as described above makes it particularly useful for holding a plurality of personal use articles 3. In particular, a user may transport a plurality of such articles 3 in her purse or the like and at the same time maintain the articles in a clean, secure condition.

The releasable attachment of the longitudinal edges 16, 17 of the front wall 11 and rear wall 12 of each wrap 10 can be achieved, such as by using an adequate adhesive applied to one of the abutting surfaces of the overlapped portions of material located adjacent to one of said longitudinal edges 16, 17.

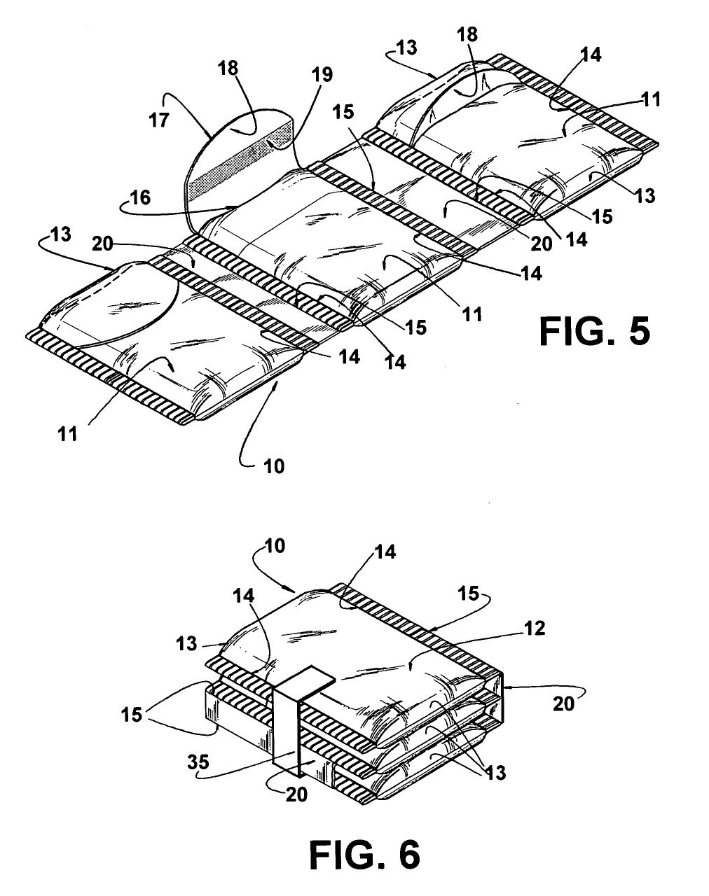

FIGS. 5 and 6 refer to a second embodiment of the invention. In the embodiment shown in FIGS. 5 and 6 the front wall 11 terminates at a longitudinal edge 16. The rear wall 12 includes a closing flap 18 that is structured and arranged to enable the closing flap 18 to be folded over at least a portion of front wall 11 to thereby seal the wrap 10.

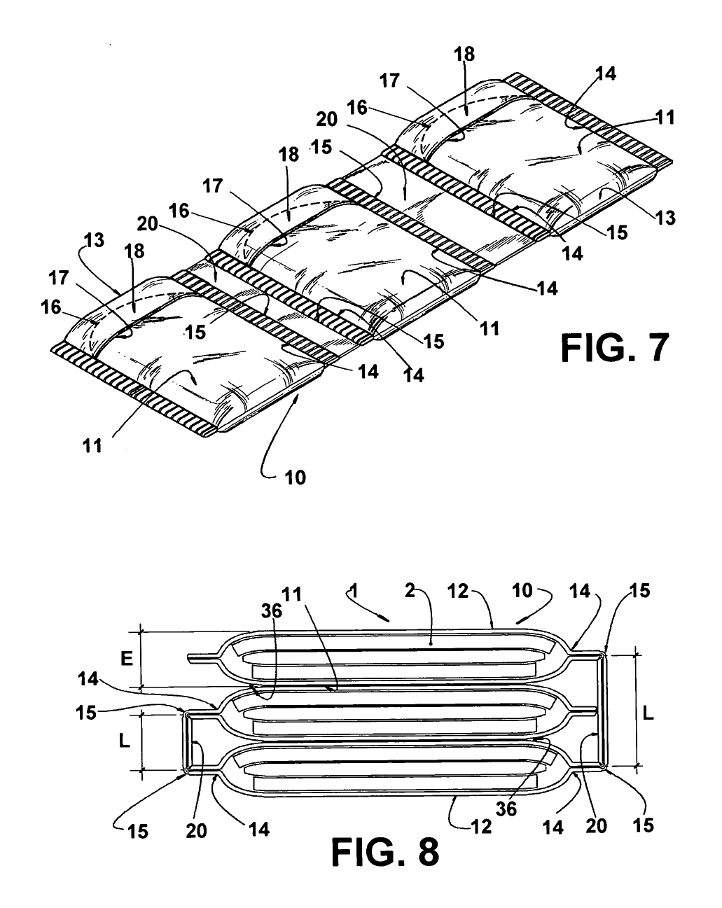

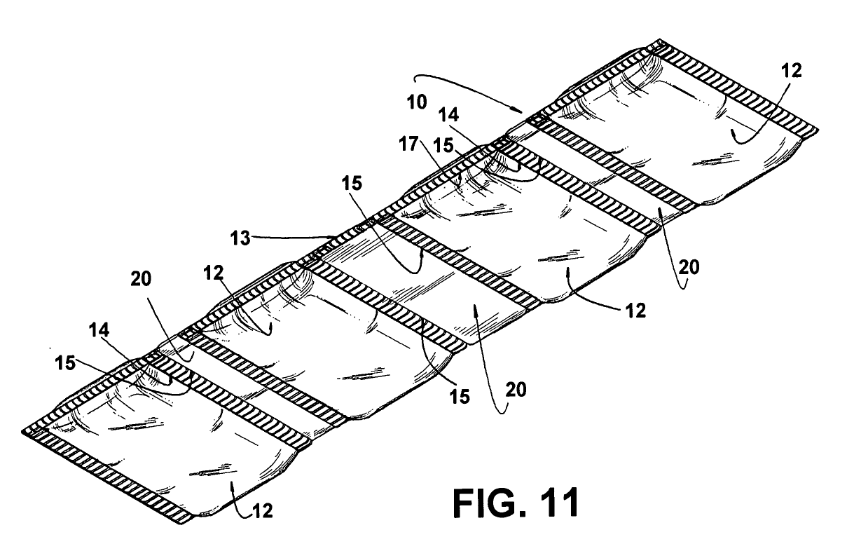

As illustrated in FIGS. 5 and 6, the closing flap 18 of the rear wall 12, as well as the front wall 11, can be provided, respectively, in their internal and/or external faces with a coating of adhesive 19, in order to guarantee a releasable and reclosable retention of the closing flap 18 on the front wall 11 of the wrap. It should be understood that, in this second embodiment illustrated in FIGS. 5 and 6, the package could contain a larger number of wraps 10 separated from each other by respective joining bridges 20, as illustrated in FIGS. 7, 8, 9, 10 and 11.

FIGS. 7 and 8 refer to a third embodiment of the invention, similar to that of FIGS. 5 and 6. In the embodiment shown in FIGS. 7 and 8, the longitudinal edge 16 of the front wall 11 of the wrap is structured to define an arc of a circle medianly tangent to the alignment of the adjacent longitudinal lateral edge 13 of the wrap 10. The rear wall 12 of the wrap is structured to include a closing flap 18 that is structured and arranged to enable the closing flap 18 to be folded over at least a portion of front wall 11 to thereby seal the wrap 10.

The third wrap embodiment described above is particularly useful to hold a plurality of personal use articles 2 therein where such articles 2 are in a flat, i.e. non-folded, state. However, the third wrap embodiment may also be used to hold folded personal user articles 2. The flap 18 and/or front wall 11 may optionally include an adhesive to enable the flap 18 to be selectively secured to the front wall or in the alternative the flap 18 may be structured to have a size sufficiently large so as to make such adhesive optional.

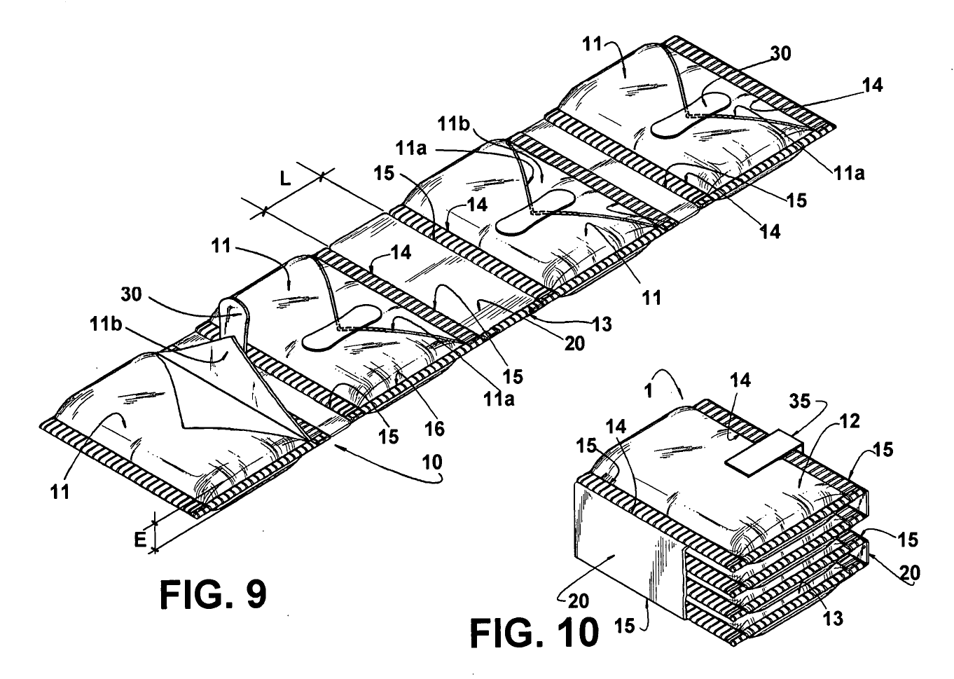

FIGS. 9, 10 and 11 refer to a fourth embodiment of the invention. In the embodiment shown in FIGS. 9-11 the edges defining peripheral side edges of each wrap 10 are sealed, and access to the interior of each wrap 10 is obtained by a generally “V” shaped cut 11a provided in the front wall 11 of each wrap 10. The cut 11a in the front wall defines a closing flap 11b, having an edge defined by a transversal end edge 14 of the wrap 10 and the opposite free edge defined by the cut 11a produced in the front wall 11 of the wrap 10. The closing flap 11b can be manually displaced by the user between a closed position, in which it remains with its free edge level with the remaining part of the front wall 11 in the region of the cutout 11a, and a raised open position, in which it permits access to the inside of the wrap 10.

In order to assure the retention of the closing portion 11b in the closed position, a lug or tab 30 may provided, which is detachably glued to the front wall 11 of each wrap 10, transversally over the cutout 11a so as to selectively maintain the closing flap 11b in the closed condition.

It should be understood that the lug 30 could be further applied to the closing flaps 18 of the wraps 10 of the embodiments illustrated in FIGS. 5-6 and 7-8 to make the closure of the wraps 10 more efficient.

In the embodiment illustrated in FIGS. 9-11, the package includes an even number of wraps 10, usually four, the wraps 10 being separated from each other by respective joining bridges 20 formed by the film that forms the wraps 10. The bridge 20 preferably has a length “L” which corresponds, approximately, to the sum of the thicknesses “E” of the wraps 10 which, when folded, are disposed on planes parallel to each other and orthogonal and secant to a respective joining flap 20. In general, it can be said that a length “L” of each joining bridge 20 is at least equal to the sum of the thicknesses “E” of the “n-1” wraps 10 that are orthogonal and secant to said joining flap 20 and not greater than the sum of the thicknesses “E” of said “n” wraps 10.

With the described construction, the outermost wraps 10 can be folded over the innermost wraps 10, until the wraps 10 occupy positions that are parallel and adjacent to each other and orthogonal and secant to the respective joining flaps 20, forming a kind of case, as illustrated in FIG. 10.

In order to maintain the wraps 10 in the folded condition illustrated in FIGS. 2, 6, 8 and 10, there can be provided a closing flap 35 for connecting, in a selectively releasable manner, the transversal end edges 14 of the two outermost wraps 10 of the case to be formed. It should be understood that the closing flap 35 could be replaced by a drop of a “hot-melt” adhesive 36 applied to the mutually seated internal faces of the wraps 10 when in the folded condition, as illustrated in FIG. 8, or by any other fixation means.

While only one way of carrying out the invention has been illustrated herein, it should be understood that changes in the form and arrangement of the different parts could be made, without departing from the constructive concept defined in the appended claims.

1. A portable package for articles of personal use, such as feminine absorbents comprising:
a strip (1) containing at least one row of wraps (10) each wrap (10) containing at least one personal use article (2),
each wrap (10) having a front wall (11) and a rear wall (12) having longitudinal lateral edges (13) and transversal end edges (14) united to each other, wherein transversal end edges (14) of two adjacent wraps (10) are coupled to each other by a joining bridge (20) in order to allow the wraps (10) to be folded one over the other and selectively separated from each other.

2. The package as set forth in claim 1, wherein said joining bridge (20) is coupled to said transversal end edges (14) of two adjacent wraps (10) along a respective fold line (15) around which the wraps (10) can be folded to occupy positions parallel to each other and orthogonal to the joining bridge (20).

3. The package as set forth in claim 2, wherein at least one fold line (15) adjacent to a joining flap (20) is structured and arranged to enable a user to manually rupture said strip (1) along said fold line (15) whereby a selected wrap (10) can be separated from the row of wraps.

4. The package as set forth in claim 2, where said joining bridge (20) has, in the longitudinal direction of the row, has a length (L), said length (L) being substantially equal to the sum of the thicknesses (E) of the wraps (10) disposed on planes parallel to each other and orthogonal and secant in relation to said joining bridge (20).

5. The package as set forth in claim 4, wherein the length (L) of each joining flap (20) is at least approximately equal to the sum of the thicknesses (E) of the “n−1” wraps (10) disposed on planes orthogonal and secant to said joining flap (20) and not greater than the sum of the thicknesses (E) of said “n” wraps (10).

6. The package as set forth in claim 4, further comprising a closing flap (35) uniting, in a selectively releaseable manner, two external wraps (10) of a plurality of wraps folded one over the other.

7. The package as set forth in claim 4, further comprising a drop of “hot-melt” adhesive (36) applied on mutually seated internal faces of the wraps (10) when in the folded condition.

8. The package as set forth in claim 1, wherein personal use articles (2) are arranged in a folded condition inside each respective wrap (10).

9. The package as set forth in claim 1, wherein the wraps (10) have their front wall (11) and rear wall (12) defined by a continuous film of packaging material, the front wall (11) and the rear wall (12) of each wrap (10) presenting longitudinal edges (16, 17) which are arranged on top of each other and releasably affixed to each other, in order to enable said wrapper to be opened and closed.

10. The package as set forth in claim 9, wherein the two longitudinal edges (16, 17) of the front wall (11) and rear wall (12) of each wrap (10) are releasably affixed to one another by an adhesive (19) applied to at least one of the abutting faces of the front wall (11) and rear wall (12) of each wrap (10).

11. The package as set forth in claim 9, wherein said rear wall (10) is structured to include a closing flap (18), said closing flap (18) being structured and arranged to be selectively folded over the front wall (11) of the same wrap (10) thereby selectively closing said wrap (10).

12. The package as set forth in claim 9, wherein the longitudinal edge (16) of the front wall (11) of a wrap (10), defined between the respective pair of transversal end edges (14), defines an arc of a circle medianly tangent to the alignment of the adjacent longitudinal lateral edge (13) of the wrap (10), the longitudinal edge (17) of the rear wall (12) of the wrap (10) being defined in a closing flap (18), which is dimensioned to be forwardly folded and seated, in superposition, over a certain extension of the front wall (11) of the wrap (10).

13. The package as set forth in claim 11, wherein at least one of the closing flap (18) and the front wall (11) is provided, adhesive (19) so as to provide a releasable and reclosable retention of the closing flap (18) on the front wall (11) of the wrap (10).

14. The package as set forth in claim 11, further comprising a lug (30) for selectively securing said closing flap (18) to the adjacent portion of the front wall (1) of the wrap (10).

15. The package as set forth in claim 1, wherein the front wall (11) is provided with a cutout (11a) defining a closing portion (11b) which is manually displaceable between a closed position and a raised opened position permitting access to the interior of the wrap (10).

16. The package as set forth in claim 15, further comprises a lug (30) coupled to said cutout (11a) said lug (30) being structured and arranged to selectively maintain said closing portion (11b) in said closed position.
